(19) United States
(12) Patent Application Publication
(10) Pub. No.: US 2020/0323420 A1
(43) Pub. Date: Oct. 15, 2020

(54) ENDOSCOPIC MULTI-TOOL

(71) Applicant: THE HOSPITAL FOR SICK CHILDREN, Toronto (CA)

(72) Inventors: Gloria WU, Markham (CA); Marko MIKIC, Toronto (CA); Kevin Aisin GIORO, Toronto (CA); Thomas LOOI, Markham (CA); James DRAKE, Toronto (CA); Alexander ALVARA, Berkeley, CA (US); Arushri SWARUP, Kitchener (CA)

(21) Appl. No.: 16/847,235

(22) Filed: Apr. 13, 2020

Related U.S. Application Data
(60) Provisional application No. 62/833,549, filed on Apr. 12, 2019.

(51) Int. Cl. A61B 1/00; A61B 1/018; A61B 1/005; A61B 1/015

(52) U.S. Cl. CPC A61B 1/00098; A61B 1/018; A61B 1/0052; A61B 1/00137; A61B 1/0057; A61B 1/00133; A61B 1/015; A61B 1/00135

(57) ABSTRACT

A hand-held endoscopic multi-tool includes a dextrous hollow tube, a cap, a main body, a tool body, an endoscope and a tool. The dextrous hollow tube has a generally rigid portion at the proximal end and a bendable portion at the distal end. The cap is operably attached to the distal end of the dextrous hollow tube and the main body to the proximal end. The tool body is moveably connected to the main body and moveable in a linear direction relative to the main body and co-axial to the tube. The endoscope is housed within the tube and operably attached to the cap. The tool is housed within the tube and constrained by the cap such that the tool is operably coupled to the movement of the tube and the tool is operably attached to the tool body whereby movement of the tool body moves the tool.

### FIELD OF THE DISCLOSURE

This disclosure relates to endoscopic tools and in particular endoscopic multi-tools.

### BACKGROUND

An endoscope is a medical device for insertion into a body passageway or cavity that enables an operator, positioned at a remote external location, to perform certain surgical procedures at a site internal to the patient's body. In general, an endoscope includes a long, sometimes flexible tubular member equipped with, for example, a miniature viewing device and an illumination device. The endoscope has a proximal end that remains external to the patient and a distal end having an endoscope tip for insertion into a body cavity of the patient. The size and rigidity of commonly available endoscopes occupy a large portion of restricted surgical working channels and cannot access certain anatomical structures due to the requirement for bending. While flexible endoscopes are available, they lack precise positioning and orientation due to their flimsy nature.

### SUMMARY

A hand-held endoscopic multi-tool includes a dextrous hollow tube, a cap, a main body, a tool body, an endoscope and a tool. The dextrous hollow tube has a generally rigid portion at the proximal end and a bendable portion at the distal end. The cap is operably attached to the distal end of the dextrous hollow tube. The main body is operably attached to the proximal end of the dextrous hollow tube. The tool body is moveably connected to the main body and moveable in a linear direction relative to the main body and co-axial to the dextrous hollow tube. The endoscope is housed within the dextrous hollow tube and operably attached to the cap. The tool housed within the dextrous hollow tube and constrained by the cap such that the tool is operably coupled to the movement of the dextrous hollow tube and the tool is operably attached to the tool body whereby movement of the tool body moves the tool.

The main body may include a tip articulation mechanism operably attached to the dextrous hollow tube such that the bendable portion of the dextrous hollow tube bends responsive to movement of the tip articulation mechanism.

The tip articulation mechanism may include a plurality of cables attached to the distal end of the dextrous hollow tube such that an applied force in the distal direction causes deformation.

The tip articulation mechanism may include a roll mechanism such that activating the roll mechanism causes rotation of the dextrous hollow tube. The tip articulation mechanism may be a motorized tip articulation mechanism.

The tool may contain additional degrees of freedom.

The hand-held endoscopic multi-tool may further include a separate component operably attached to both the tool body and the tool, such that the component can rotate independently to the tool body but is rotationally coupled with the tool.

The hand-held endoscopic multi-tool may further include a plurality of cables operably attached to the distal end of the tool such that an applied force in the distal direction causes deformation of the distal end of the tool.

The hand-held endoscopic multi-tool may further include at least one hollow tube housed within the tube and operably attached to the cap. The at least one hollow tube may be the tube portion of a suction and irrigation system.

A secondary tool may be inserted into the at least one hollow tube. The secondary tool may be manipulated in multiple degrees of freedom. The secondary tool may further include a plurality of cables operably attached to the distal end of the tool such that an applied force in the distal direction causes deformation of the distal end of the tool. The secondary tool may be rotatable wherein roll is achieved by physically turning the secondary tool.

The hand-held endoscopic multi-tool may have a plurality of additional tools inserted through the cap and controlled by their respective tool bodies.

Further features will be described or will become apparent in the course of the following detailed description.

### BRIEF DESCRIPTION OF THE DRAWINGS

The embodiments will now be described by way of example only, with reference to the accompanying drawings, in which:

FIG. 1 is a side view of an endoscopic multi-tool;

FIG. 2A is a perspective view of the tip of the endoscopic multi-tool of FIG. 1;

FIG. 2B is a side view of the tip of FIG. 2A and the capstan assembly of the endoscopic multi-tool of FIG. 1;

FIG. 2C is a side view of the tip and the capstan assembly similar to that shown in FIG. 2A but showing the tip in a bent position;

FIG. 3 is a side view of a portion of the endoscopic multi-tool of FIG. 1 and showing the actuation mechanism without supports;

FIG. 4A is a side view of the tip and the dissector extension and retraction mechanism of the endoscopic multi-tool of FIG. 1;

FIG. 4B is a side view of the tip and the dissector extension and retraction mechanism of FIG. 4A shown in an extended position and showing the internal tubing;

FIG. 4C is an exploded perspective view of the dissector extension and retraction mechanism of FIGS. 4A and 4B;

FIG. 5 is a side view of the dissector extension and retraction mechanism of FIG. 4A inside the full assembly of the endoscopic multi-tool;

FIG. 6 is a schematic view of the suction and irrigation system of the endoscopic multi-tool of FIG. 1;

FIG. 7 is a side view of an alternate embodiment of the endoscope multi-tool having an alternate tip articulation mechanism;

FIG. 8A is an enlarged side view of the tip articulation mechanism and the tool mechanism of the endoscope multi-tool of FIG. 7;

FIG. 8B is an enlarged sectional view of only the tip articulation mechanism shown in FIG. 8A;

FIG. 9A is an enlarged sectional view of the tip articulation mechanism similar to that shown in FIG. 8B

FIG. 9B is an enlarged sectional view of the tip articulation mechanism from FIG. 9A;

FIG. 10A is a partial perspective view of a dexterous hollow tube of the endoscope multi-tool of FIG. 7;

FIG. 10B is a partial perspective view of a dexterous hollow tube similar to that shown in FIG. 10A but showing the tip of the hollow tube bent;

FIG. 10C is a partial perspective view of a dexterous hollow tube similar to that shown in FIG. 10B but showing the tip bent in a direction perpendicular to that of 10B;

FIG. 11A is an enlarged side view of the tip articulation mechanism and the instrument articulation mechanism of the endoscope multi-tool of FIG. 7, similar to that shown in FIG. 8A;

FIG. 11B is an enlarged sectional view taken from FIG. 11A in the opposite direction of FIG. 8B and shows the instrument articulation mechanism which is part of the tool body;

FIG. 12A is a side view of an alternate endoscopic multi-tool showing the tool in the extended position;

FIG. 12B is a side view of the alternate endoscopic multi-tool showing the tool in the retracted position;

FIG. 13A is a side view of a suction-irrigation tube for use in the endoscopic multi-tool;

FIG. 13B is a side view similar to that shown in FIG. 13A but showing an alternate tool inserted in the suction-irrigation tube of FIG. 13A;

FIG. 14A is a sectional view of FIG. 13B with the tool body connected through the gear to the instrument; and

FIG. 14B is a sectional view of FIG. 13B with the tool body operably connected to the instrument but showing the instrument tip bent.

### DETAILED DESCRIPTION

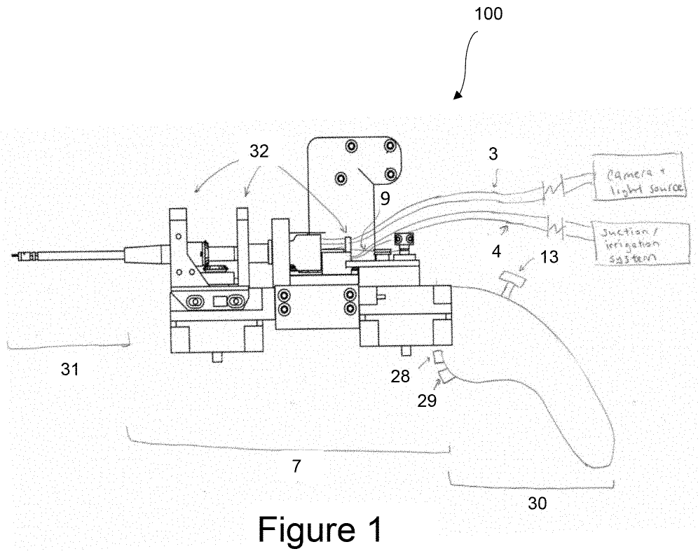

Referring to FIG. 1, the endoscopic multi-tool is shown generally at 100. Multi-tool 100 is a handheld, electromechanical instrument for minimally invasive surgery. Multi-tool 100 includes a steerable multifunctional tip 31, a base or main body 7 and a handle 30.

Endoscopic multi-tool 100 is a surgical device that consists of a steerable multifunctional tip. Endoscopic multi-tool 100 is capable of performing suction/irrigation, vision, and dissection, where the dissector can be manually translated into and out of the tube, and the outer tube is able to roll and pitch as required. The articulation (roll and pitch), suction and irrigation functions are electromechanically activated.

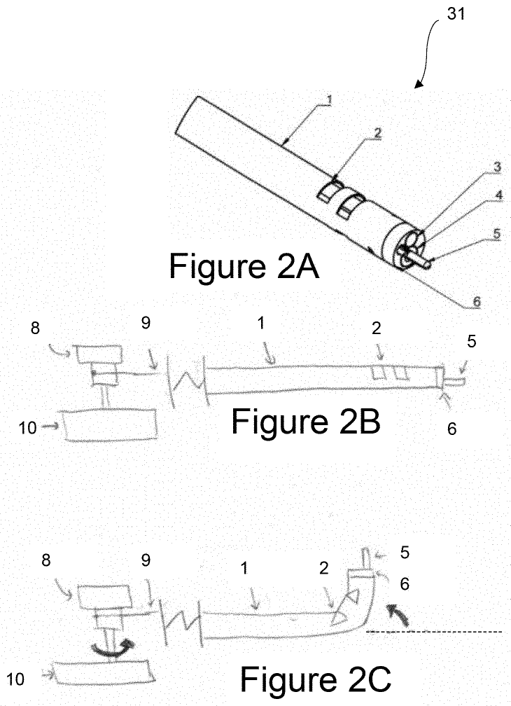

Tip 31 is best seen in FIGS. 2A to 2C. Tip 31 has an outer tube 1. The outer tube shown herein has diameter of 4mm and is preferably made of nitinol. Nitinol is a nominally rigid material that has super elastic properties. The outer tube 1 has a plurality of geometrical cuts 2 formed therein to allow the tube 1 to bend. It will be appreciated by those skilled in the art that other materials may also be used for the outer tube 1. In some embodiment, the outer tube may have the requisite rigidity, flexibility and bendability such that the geometrical cuts may not be needed.

A plurality of items is enclosed within the tube 1 of the tip 31. For example, a waterproof flexible endoscope/camera on a chip system 3; a flexible tube 4 for suction and irrigation; and a rigid dissector tip are housed within the tube 1 of the tip 31. The tool 5 may be surgical tool with a functional end effector. The tool 5 may be ˜1 mm in diameter and may include graspers, cautery, scalpel, needles etc, all of which are attached to a tool flexible body. A cable 9 runs through the tube 1 and is attached to the distal end of the tip such that the tip will bend responsive to the cable being pulled. A cap 6 is operably attached to the outer tube 1 and ensures that the suction/irrigation tube 4, endoscope, and tool 5 is rotationally coupled to the cap 6 such that when the tube 1 and cap 6 rotate the items therein also rotate. Tool 5 is held in the cap 6 such that it can easily translate into and out of the cap 6 such that the tool can be extended or retracted from the cap 6. Tube 1 is operably attached to the base 7.

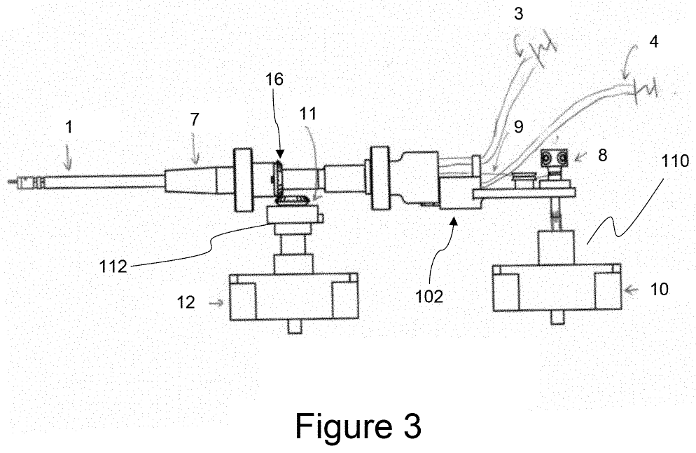

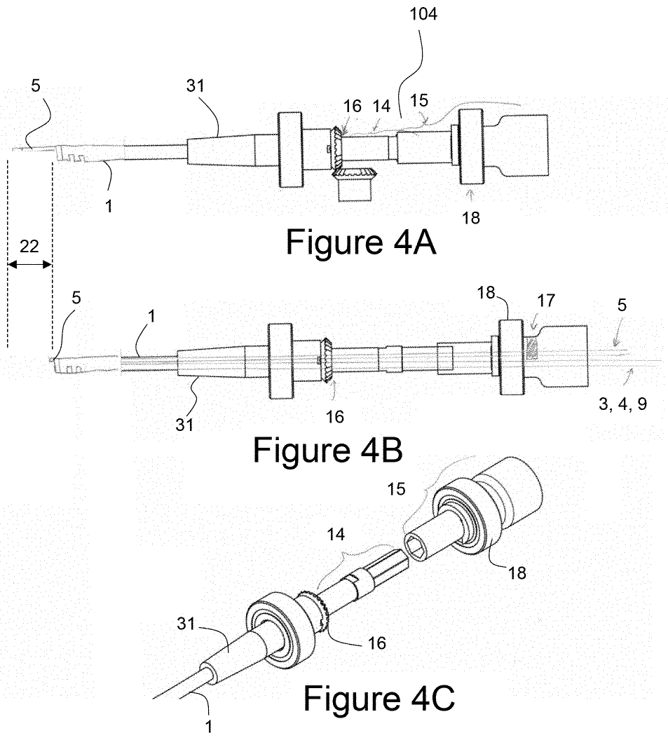

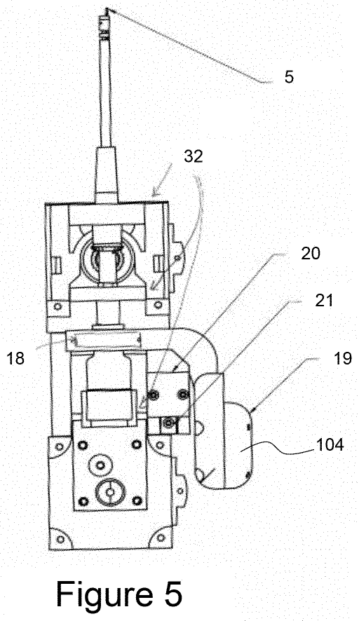

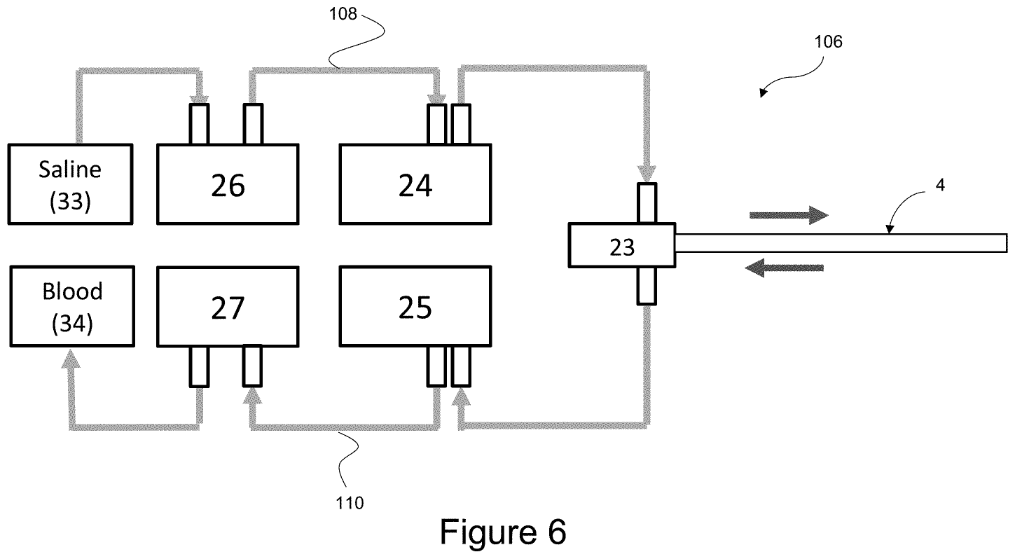

Base 7 includes a tip articulation mechanism 102 (best seen in FIG. 3), a tool translation assembly 104 (best seen in FIGS. 4A to C and FIG. 5) and a suction and irrigation system 106 (best seen in FIG. 6).

The tip articulation mechanism 102 includes an electromechanical pitch mechanism 110 and an electromechanical roll mechanism 112. The electromechanical pitch mechanism 110 includes a cable 9 that is operably connected to a capstan 8 that is driven by a motor 10. Activation of the motor 10 will pull the cable 9, close the notches 2 and pitch (or bend) of the outer tube 6 and its contents as best seen in FIG. 2C.

The outer tube 1 of tip 31 is operably connected to a pair of bevelled gears 11, 16 for 90 degree motion transfer that is driven by a motor 12. Activation of the motor 12 will rotate bevelled gear 11, which in turn rotates bevelled gear 16 thus activation of the motor 12 will roll (spin) the tip 31 and its contents. Bevelled gear 16 is operably attached to the outer tube 6. Bevelled gear 16 is hollow and allows the flexible instruments to run therethrough.

The motors 10, 12 are activated by the user through a joystick 13, where each axis controls a different motor. The joystick 13 is located on a handle 30 at the back of the instrument as shown in FIG. 1.

Tool translation assembly 104 is shown in FIGS. 4A to C and FIG. 5. Tool translation assembly 104 includes an inner hex portion 14 and an outer hex portion 15. As shown in FIG. 4C the inner hex portion 14 can freely slide in and out of the outer hex portion 15, but the inner hex portion 14 is rotationally fixed relative to the outer hex portion 15. The inner and outer hex portions are coaxial to the outer tube 1. The inner hex portion 14 piece is operably attached to the bevelled gear 16 that is operably attached to the outer tube 1 and can rotate with the outer tube and bevelled gear system. The flexible instruments can run through the inner hex portion 14. Tool 5 is operably attached to the outer hex portion 15 at 17. Tool 5 does not need to be centered within outer hex portion 15. The other flexible instruments run through the outer hex portion 15 and are not attached thereto. A bearing 18 is positioned distally, spaced from but concentric to the outer tube 1. The outer hex portion 15 is operably attached to the inside of the bearing 18 and concentric to it. Pushing on the bearing 18 in the axial direction of the bearing will translate the outer hex piece as well. A pusher handle 19 (shown in FIG. 5) is operably attached to the outside of the bearing 18 such that the direction of force applied to push the handle 19 is parallel to the outer tube 1. Pushing on the handle 19 will push the bearing 18 in its axial direction.

Supports 32 operably attached the tool translation assembly 104 to the tip articulation mechanism 102. Supports 32 are attached to the tool translation assembly 104 such that the inner hex portion 14 is free to rotate with the outer tube 1 but is otherwise constrained. The outer hex portion 15 is free to slide coaxially inwardly and outwardly relative to the outer tube 1 and free to rotate with the outer tube 1, but is otherwise constrained. The handle 19 is supported by a carriage 20 and rail 21 or translational bearing mechanism, which also allows it to easily slide forwardly and backwardly (parallel to the outer tube 1). Referring to FIGS. 4A and 4B, FIG. 4A shows the tool 5 extended and FIG. 4B showing the tool 5 retracted. The translation distance between the retracted position and the extended position is shown at 22.

The suction and irrigation system is shown generally at 106 in FIG. 6. The suction and irrigation tube 4 is connected to a splitter 23. The splitter 23 splits into a saline portion 108 and a blood portion 110. The saline portion 108 includes a saline chamber 33 in flow connection with a saline pump 26 and a saline valve 24, and is in flow connection with one side of the splitter 23. Similarly, the blood portion 110 includes a blood chamber 34 in flow connection with a suction pump 27 and a suction valve 25, and is in flow connection with the other side of the splitter 23. The saline portion 108 is for irrigation and the blood portion 110 is for suction. Saline pump 26 is operably connected to a saline button 28 on handle 30 (FIG. 1) and saline valve 24 and suction valve 25. Suction button 29 is operably connected to suction pump 27 and saline valve 24 and suction valve 25. Pressing one of two buttons (28-29) will activate the relevant pump, open one valve and shut the other so that the suction and irrigation are separate.

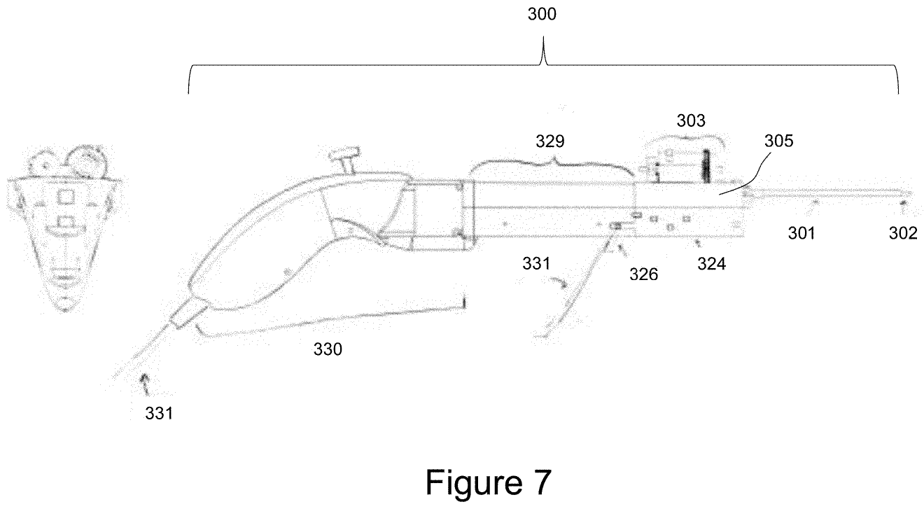

An alternative embodiment of the design 300 is shown in FIGS. 7 to 11. The outer tube 301 is shown operably attached to the main body 305. The instrument 302 is operably attached to the tool body 303. A handle 330 is operably attached to the main body 305. The tool body 303 has a carriage 340 which runs on a rail 304 that is operably attached to the main body 305, such that the tool body can slide coaxial to the outer tube 301 axis. Therefore, when the tool body 303 is moved forwards, the instrument 302 can extend past the distal tip of the outer tube 301 and also retract when the tool body moves backwards in the axial direction.

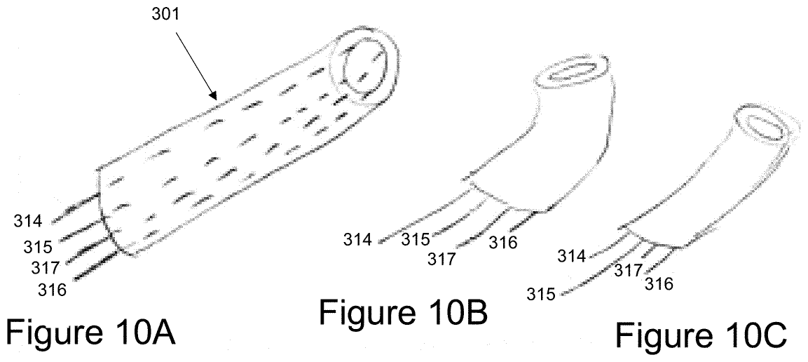

An alternative tip articulation mechanism for the dextrous hollow tube 301 bending is shown in FIGS. 7 to 11. As shown in FIGS. 10A to 10C four cables (314, 315, 315, 317) are attached to the distal tip of the outer tube 301. Two pairs of cables form an opposing set (315 with 317, and 314 with 316) that can bend the tube along one plane in two opposite directions (see 10B and 10C).

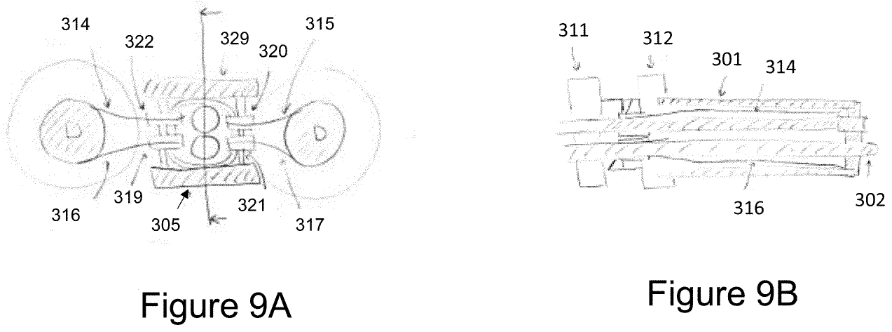

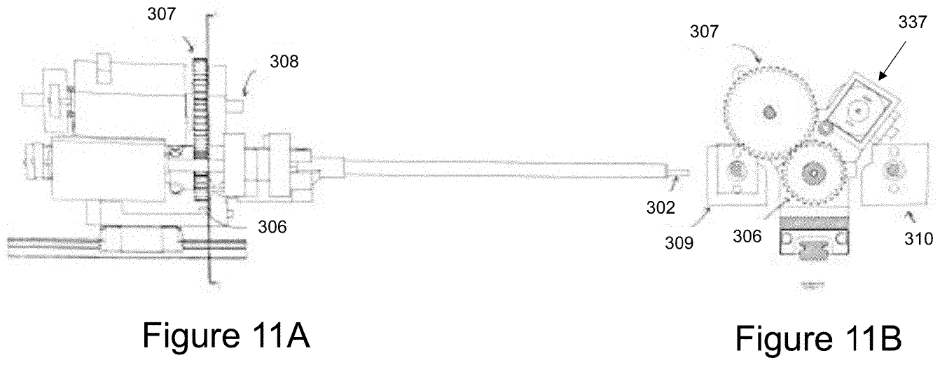

In this alternative design 300, each cable is threaded through the outer tube 301, around a pulley and then operably attached to a capstan (311, 312, 313, 318) shown in FIGS. 9A and 9B. The pairs of opposing capstans are rotationally coupled to a motor (310 and 309 shown in FIG. 11B) such that the rotation of a motor in one direction, will pull one of the cables and loosen the opposing cable thus bending the outer tube 301 in the desired direction.

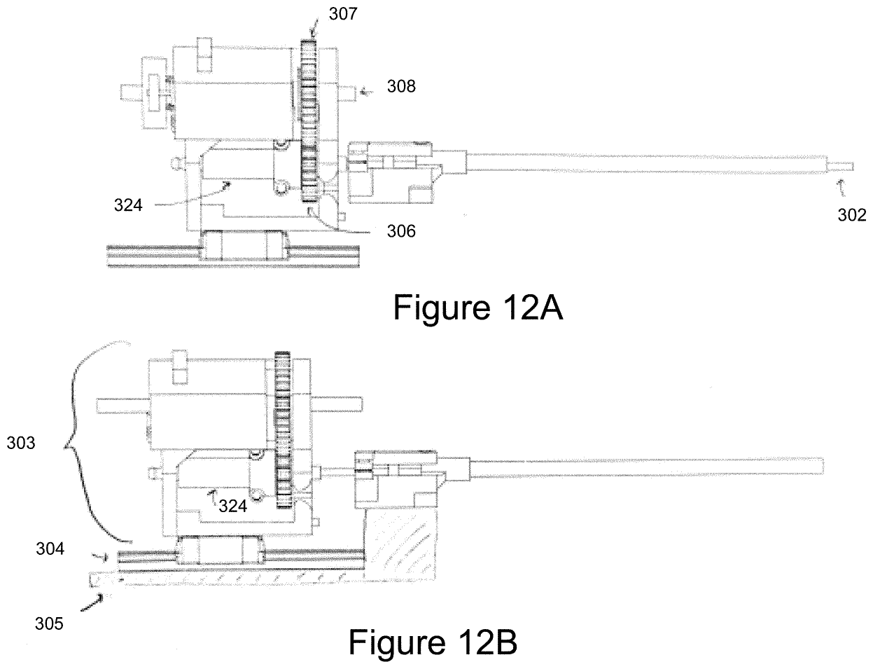

Optionally, the instrument 302 is configured to roll relative to the tool body as shown in FIGS. 12 to 14. The instrument 302 is operably attached to a gear 306, which is coupled to a second gear 307. The second gear is mounted onto a motor shaft which drives said second gear 307, which drives the first gear 306 and the instrument 302. Alternatively, the second gear can be manually spun by the user if a spin handle is included.

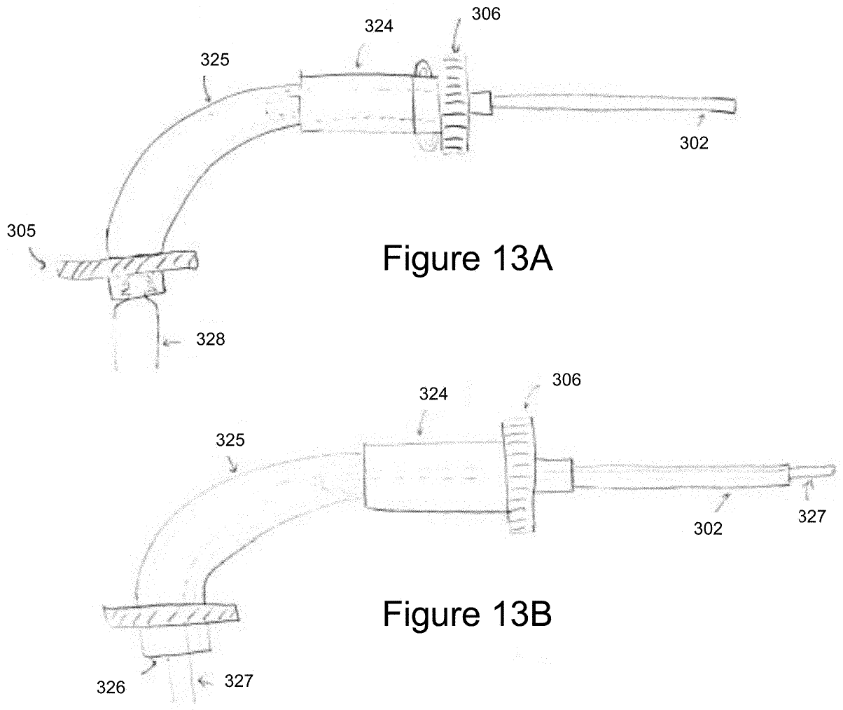

In this alternative embodiment 300, the instrument 302 is a hollow tube which allows for a number of functions. For example, the hollow instrument 302 can be a suction-irrigation tube as shown in FIG. 13A. The chamber has an outlet that a flexible tube 325 can plug onto which provides a good seal. This flexible tube 325 can then be directed out of the main body 305 of the device. Then a suction-irrigation system as shown in FIG. 6 can be connected to this flexible tube 325.

In a second example, an existing flexible tool 327 can be threaded through the flexible tube 325, the chamber 324, the gear 306, and then the instrument 302, shown in FIG. 13B. This flexible tool 327 could be but is not limited to graspers, a biopsy needle, cautery, forceps and other flexible surgical tools.

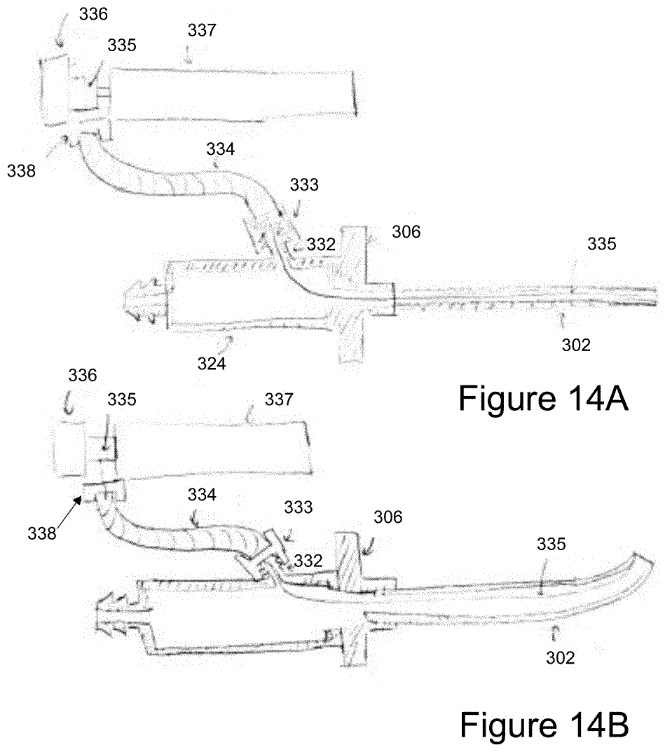

Furthermore, the instrument 302 can also be a dextrous hollow tube that has a bendable portion at its distal end. Then a cable 335 attached to the distal end of the instrument can be pulled by a capstan 336 attached to a motor 337 to bend the instrument 302 (see FIGS. 14A and B). In the case that suction and irrigation are required, the cable goes through a seal, for example a gasket 333 when it exits the suction-irrigation chamber 324.

In the case that the instrument 302 can roll, the chamber 324 will also roll. Therefore, the cable is inserted into a sheath 334 that is operably attached to the chamber by 333 and the motor/capstan 338 such that the sheath maintains the cable length so that the cable can maintain tension at any position, as in a bike break.

The endoscopic multi-tool 100 is a handheld, electromechanical instrument for minimally invasive surgery, composed of a steerable multifunctional tip. The endoscopic multi-tool 100 includes a 2-DOF tube 1 that houses several dextrous components including but not limited to: suction/irrigation tube, endoscope, and swappable dissector (blunt/grippers/scalpel/biopsy forceps etc.).

The outer tube 1 is defined as a small (sub 4 mm) hollow tube with thin walls (to maximize inner diameter and minimize outer diameter) that is nominally rigid, but bendable in certain directions. This flexibility can be achieved in several ways:

a) Tube made of a nitinol (super-elastic nickel titanium) tube with geometric cuts that remove material, allowing the tube to bend when cable tension is applied. Original position is recovered when the tension is released (also known as a continuum wrist) as shown in FIGS. 1 and 3;

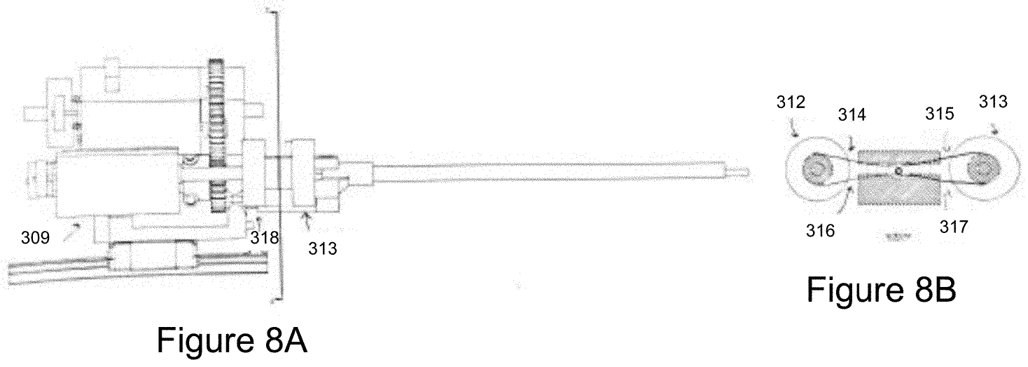

b) Tube made of a stainless-steel tube with a spiral cut that allows it to bend in multiple directions with an applied cable tension as shown in FIGS. 7 and 8.

The tube 1 is operably attached to the main body 7 of the instrument 100 and is cable actuated. Each cable is attached to the distal end of the tube 1 and runs along the length of the tube. Pulling on the cables will pull on the tube, and since the tube is fixed into the main body 7, the force applied will cause the tube to bend in the direction that the cable is pulling. Each cable is wrapped onto a capstan that is rotated by a motor that is also operably attached to the main body of the instrument.

Each motor is activated by a joystick 13, for which the directions depend on the actuation type. Roll and pitch are controlled by horizontal and vertical joystick 13 motion, respectively. For pitch and yaw, the joystick 13 is directly mapped to the desired activation direction.

An inner component such as tool 5 is operably attached to a tool translation assembly, where the tool translation assembly is operably attached to the body of the instrument 100. The inner component 5 is also attached to a tool handle so that the user can manually extend or retract the inner component, allowing the user to either improve reachability or a conceal instrument during navigation.

For space optimization, a single tube can be used for suction and irrigation, allowing more components to be placed inside the outer lumen. This main line runs along the outer tube 1 and the actuation unit but is eventually split into separate one-way solenoid valves as shown in FIG. 6. Each valve leads to a pump that draws from or empties into separate reservoirs. Suction and irrigation are activated by pressing the buttons on the handle, engaging each respective pump and valve, but never at the same time. The activation can be modified from the current binary method to an analog feature, where pressing harder will proportionally increase the flow rate of the suction/irrigation.

The same steering mechanism for the outer lumen can also be applied to the dexterous internal components. For example, the existing silicone suction and irrigation tube can be replaced by notched nitinol or steel with spiral cuts, however the former would require covered notches for flow to be possible. A gasket would also be necessary for the actuation cable to pass through so that it may attach to a capstan that can be actuated with a motor.

Generally speaking, the systems described herein are directed to endoscopic tools. Various embodiments and aspects of the disclosure are described in the detailed description. The description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms, “comprises” and “comprising” are to be construed as being inclusive and open ended, and not exclusive. Specifically, when used in the specification and claims, the terms, “comprises” and “comprising” and variations thereof mean the specified features, steps or components are included. These terms are not to be interpreted to exclude the presence of other features, steps or components.

As used herein the “operably connected” or “operably attached” means that the two elements are connected or attached either directly or indirectly. Accordingly, the items need not be directly connected or attached but may have other items connected or attached therebetween.

What is claimed is:

1. A hand-held endoscopic multi-tool comprising:
a dextrous hollow tube having a generally rigid portion at the proximal end and a bendable portion at the distal end;
a cap operably attached to the distal end of the dextrous hollow tube;
a main body operably attached to the proximal end of the dextrous hollow tube;
a tool body moveably connected to the main body and moveable in a linear direction relative to the main body and co-axial to the dextrous hollow tube;
an endoscope housed within the dextrous hollow tube and operably attached to the cap; and
a tool housed within the dextrous hollow tube and constrained by the cap such that the tool is operably coupled to the movement of the dextrous hollow tube and the tool is operably attached to the tool body whereby movement of the tool body moves the tool.

2. The hand-held endoscopic multi-tool as claimed in claim 1 wherein the main body includes a tip articulation mechanism operably attached to the dextrous hollow tube such that the bendable portion of the dextrous hollow tube bends responsive to movement of the tip articulation mechanism.

3. The hand-held endoscopic multi-tool as claimed in claim 2 wherein the tip articulation mechanism includes a plurality of cables attached to the distal end of the dextrous hollow tube such that an applied force in the distal direction causes deformation.

4. The hand-held endoscopic multi-tool as claimed in claim 2 wherein the tip articulation mechanism includes a roll mechanism such that activating the roll mechanism causes rotation of the dextrous hollow tube.

5. The hand-held endoscopic multi-tool as claimed in claim 2 wherein the tip articulation mechanism is a motorized tip articulation mechanism.

6. The hand-held endoscopic multi-tool as claimed in claim 1 wherein the tool contains additional degrees of freedom.

7. The hand-held endoscopic multi-tool as claimed in claim 6 further including a separate component operably attached to both the tool body and the tool, such that the component can rotate independently to the tool body but is rotationally coupled with the tool.

8. The hand-held endoscopic multi-tool as claimed in claim 6 wherein the tool further includes a plurality of cables operably attached to the distal end of the tool such that an applied force in the distal direction causes deformation of the distal end of the tool.

9. The hand-held endoscopic multi-tool as claimed in claim 1 further including at least one hollow tube housed within the dextrous hollow tube and operably attached to the cap.

10. The hand-held endoscopic multi-tool as claimed in claim 9 wherein the at least one hollow tube is a tube portion of a suction and irrigation system.

11. The hand-held endoscopic multi-tool as claimed in claim 9 wherein a secondary tool is inserted into the at least one hollow tube.

12. The hand-held endoscopic multi-tool as claimed in claim 11 wherein the secondary tool can be manipulated in multiple degrees of freedom.

13. The hand-held endoscopic multi-tool as claimed in claim 12 wherein the secondary tool further includes a plurality of cables operably attached to the distal end of the tool such that an applied force in the distal direction causes deformation of the distal end of the tool.

14. The hand-held endoscopic multi-tool as claimed in claim 12 wherein the secondary tool is rotatable and roll is achieved by physically turning the secondary tool.

15. The hand-held endoscopic multi-tool as claimed in claim 1 wherein a plurality of additional tools may be inserted through the cap and controlled by their respective tool bodies.
